(19) United States
(12) Patent Application Publication
(10) Pub. No.: US 2010/0311961 A1
(43) Pub. Date: Dec. 9, 2010

(54) PROCESS AND INTERMEDIATES FOR THE PREPARATION OF SUBSTITUTED 1,3-OXATHIOLANES, ESPECIALLY LAMIVUDINE

(75) Inventors: Dinesh Shashidharan Nair, Vadodara (IN); Bishwa Prakash Rai, Azamgarh (IN); Hashim Nizar Poovanathil Nagoor Meeran, Pathanamthitta (IN); Neera Tewari, Gurgaon (IN)

Correspondence Address: Ranbaxy Inc., Intellectual Property Department, 600 College Road East, PRINCETON, NJ, 08540, US

(73) Assignee: RANBAXY LABORATORIES LIMITED, New Delhi, Delhi (IN)

(21) Appl. No.: 12/745,048

(22) PCT Filed: Apr. 30, 2008
(86) PCT No.: PCT/IB08/51691

§ 371 Date: Jul. 15, 2010

(30) Foreign Application Priority Data
Nov. 29, 2007 (IN) 2502/DEL/2007

(51) Int. Cl. C07H 19/06; C07D 327/04

(52) U.S. Cl. 536/27.13; 536/28.5; 549/30

(57) ABSTRACT

The present invention relates to process and intermediates for the preparation of substituted 1,3-oxathiolanes. The present invention specifically relates to a process for the preparation of lamivudine.

### FIELD OF THE INVENTION

The present invention relates to process and intermediates for the preparation of substituted 1,3-oxathiolanes. The present invention specifically relates to a process for the preparation of lamivudine.

### BACKGROUND OF THE INVENTION

Substituted 1,3-oxathiolanes of Formula I and stereoisomers thereof,

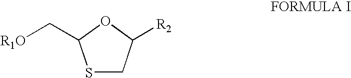

wherein R₁ is hydrogen, alkyl or aryl, and R₂ is a optionally substituted purine or pyrimidine base or an analogue or derivative thereof, are an important class of therapeutic agents and they have shown antiviral activity against retroviruses such as human immunodeficiency virus (HIV), hepatitis B virus (HBV) and human T-lymphotropic virus (HTLV). Lamivudine is a substituted 1,3-oxathiolane and it is presently available in the market as an antiretroviral agent. Lamivudine is a cis-(−)-isomer and it is chemically (2R,cis)-4-amino-1-(2-hydroxymethyl-1,3-oxathiolan-5-yl)-(1H)-pyrimidin-2-one of Formula I (A) having the structure as depicted below.

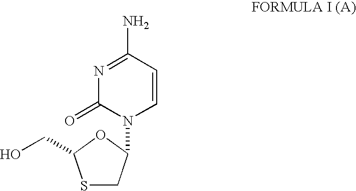

There are two different approaches provided in the prior art for preparing 1,3-oxathiolanes by using specific leaving groups and Lewis acid catalysts.

The first approach involves condensing an intermediate of Formula II, or its stereoisomers thereof

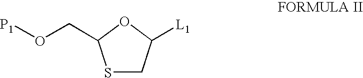

wherein P₁ is a protecting group, L₁ is OCH₃, OC₂H₅ or OCOCH₃, with a silyl and/or acetyl protected pyrimidine or purine base. The condensed product is finally deprotected to obtain desired substituted 1,3-oxathiolanes. This approach is provided in U.S. Pat. Nos. 5,047,407 and 5,905,082, J. Org. Chem., (1992), 57:2217-2219, J. Med. Chem., (1993), 36:181-195, and J. Org. Chem., (1991), 56:6503-6505. According these prior art references, the condensation is carried out in the presence of silyl Lewis acids such as trimethylsilyl triflate. However, this approach does not provide optically pure 1,3-oxathiolanes and preparation of lamivudine by this way results in a mixture of at least two of the following isomers.

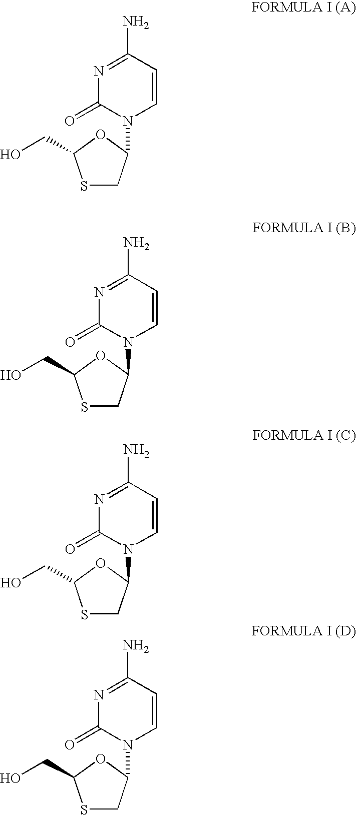

The prior art references mentioned above employ chiral chromatography, or enzymatic resolution to isolate lamivudine of Formula I (A) from said mixture. Synthetic Communications, (2002), 32:2355-2359 provides a separation method for lamivudine using a chiral auxiliary from its mixture with the compound of Formula I (B). U.S. Pat. No. 5,204,466 employs stannic chloride instead of silyl Lewis acids in the condensation of the intermediate of Formula II with silylated cytosine. However, J. Org. Chem., (1992), 57:2217-2219 says that the use of stannic chloride as a catalyst also results in a racemic mixture based on optical rotation and chiral HPLC analysis of the product obtained.

The second approach involves condensing an intermediate of Formula III, or its stereoisomers thereof

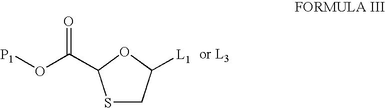

wherein P₁ is a protecting group, L₂ is OCOCH₃, L₃ is halo, with a silyl and/or acetyl protected pyrimidine or purine base. The condensed product is finally reduced and deprotected to obtain desired substituted 1,3-oxathiolanes. U.S. Pat. No. 5,663,320 provides a method to carry out the condensation in the presence of silyl Lewis acids such as iodotrimethyl silane. The U.S. '320 patent employs the compound of Formula III with OCOCH₃ group at 5-position (L₂) as an intermediate. The U.S. '320 patent further mentions that the choice of silyl Lewis acids is the key feature of the above process. However, Tetrahedron Letters, (2005), 46:8535-8538 says that said approach involving iodotrimethyl silane is proved to be inefficient for preparing lamivudine as it is low yielding and requires selective crystallization of the intermediates to obtain desired optical purity. To overcome such purity and yield issues related to Lewis acid catalysts, Tetrahedron Letters, (2005), 46:8535-8538 and U.S. Pat. No. 6,329,522 provide a method to carry out said condensation in the absence of any Lewis acid catalyst and it is achieved by selectively using the compound of Formula III with chloro substitution at 5-position (L₃) as an intermediate. Tetrahedron Letters, (2005), 46:8535-8538 reports that the chloro group was chosen as the leaving group because it provided better yield and selectivity when compared to other leaving groups. However, the preparation of the compound of Formula III with a chloro substitution at 5-position requires the use of corrosive reagents like thionyl chloride and huge quantity of dichloromethane solvent.

### SUMMARY OF THE INVENTION

We have surprisingly found that substituted 1,3-oxathiolanes, preferably lamivudine can be prepared without using Lewis acids in the condensation step even if the compound of Formula III does not have a chloro substitution at 5-position. This process provides substituted 1,3-oxathiolanes in better yield with high optical and chemical purity. We have also prepared a novel intermediate of Formula III or its stereoisomers thereof,

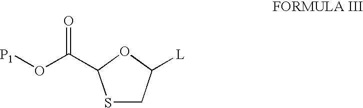

wherein P₁ is hydrogen or a protecting group and L is

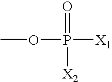

wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl, which can be efficiently used in the preparation of substituted 1,3-oxathiolanes. The present process is also suitable to prepare lamivudine at industrial scale.

The term “purine or pyrimidine base or an analogue or derivative thereof” in the present invention refers to a purine or pyrimidine base, which may be found in native nucleosides or a synthetic analogue or derivative thereof that mimics or are derived from such bases in their structures, which may either possess additional functional properties of native bases or lack certain functional properties of native bases. The analogues or derivatives include but not limited to those compounds derived by replacement of a CH₂ moiety by a nitrogen atom or replacement of a nitrogen atom by a CH₂ moiety, or both, or those compounds wherein the ring substituents are either incorporated, removed or modified by conventional substituents known in the art. The functional groups of purine or pyrimidine base or an analogue or derivative thereof may also be protected with hydroxy, amino or carboxyl protecting groups.

The term “protecting group” in the present invention refers to those used in the art and serve the function of blocking the carboxyl, amino or hydroxyl groups while the reactions are carried out at other sites of the molecule. Examples of a carboxyl protecting group include, but not limited to, optionally substituted alkyl, optionally substituted alkenyl, optionally substituted aralkyl, optionally substituted aryl, optionally substituted amino, hydrocarbonated silyl, hydrocarbonated stannyl, and a pharmaceutically active ester forming group. Examples of hydroxyl and amino protecting groups include, but not limited to, lower alkylsilyl groups, lower alkoxymethyl groups, aralkyl groups, acyl groups, lower alkoxycarbonyl groups, alkenyloxycarbonyl groups and aralkyloxycarbonyl groups. Examples of amino protecting groups include but not limited to alkylidene groups substituted with optionally protected hydroxy groups. The hydroxyl or carboxyl protecting groups can also be chiral auxiliaries, which may possess one or more chiral centers.

The term “leaving group” in the present invention refers to an atom or a group which is displaceable upon reaction with a purine or pyrimidine base or an analogue or derivative thereof. Examples of leaving groups include but not limited to optionally substituted, saturated or unsaturated acyloxy groups, alkoxy groups, alkoxy carbonyl groups, amido, azido, isocyanato, optionally substituted, saturated or unsaturated thiolates, and optionally substituted seleno, seleninyl or selenonyl. Examples of leaving groups also include but not limited to —OX, wherein X is optionally substituted aryl, heteroaryl, phosphonate, or sulfinyl group.

### DETAILED DESCRIPTION OF THE INVENTION

A first aspect of the present invention provides a compound of Formula III or its stereoisomers thereof,

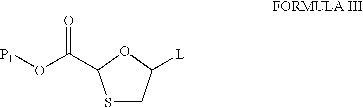

wherein P₁ is hydrogen or a protecting group and L is

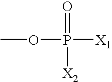

wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl. X₁ and X₂ are preferably optionally substituted aryl or aryloxy groups, and P₁ can be a chiral auxiliary.

A second aspect of the present invention provides a process for the preparation of compound of Formula III or its stereoisomers thereof,

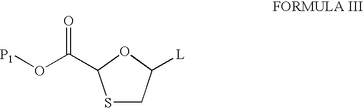

wherein P₁ is hydrogen or a protecting group and L is

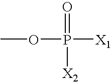

wherein X₁ and X₂ are same or different and are as defined earlier,
wherein the process comprises a step of reacting a compound of Formula V,

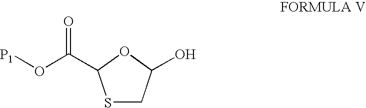

wherein P₁ is hydrogen or a protecting group, with a compound of Formula VI,

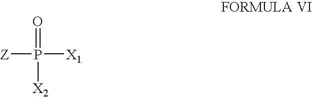

wherein X₁ and X₂ are same or different and are as defined earlier, and Z is halogen, to obtain the compound of Formula III or its stereoisomers thereof.

A third aspect of the present invention provides a process for the preparation of a substituted 1,3-oxathiolane of Formula I or its stereoisomers, and salts thereof,

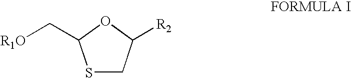

wherein R₁ is hydrogen, alkyl or aryl, and R₂ is an optionally substituted purine or pyrimidine base or an analogue or derivative thereof,
wherein the process comprises,
a) reacting a compound of Formula V,

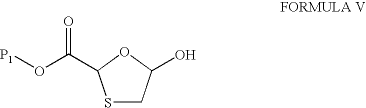

wherein P₁ is hydrogen or a protecting group, with a compound of Formula VI,

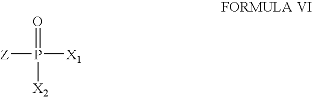

wherein X₁ and X₂ are same or different and are as defined earlier, and Z is halogen, to obtain a compound of Formula III or its stereoisomers thereof,

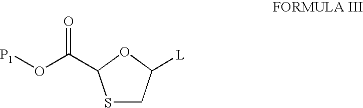

wherein P₁ is hydrogen or a protecting group and L is

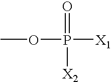

wherein X₁ and X₂ are same or different and are as defined earlier,
b) reacting a compound of Formula III or its stereoisomers thereof, with an optionally substituted purine or pyrimidine base or an analogue or derivative thereof, to obtain a compound of Formula IV or its stereoisomers thereof,

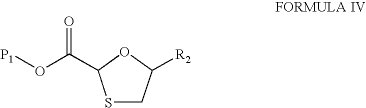

wherein P₁ is hydrogen or a protecting group and R₂ is an optionally substituted purine or pyrimidine base or an analogue or derivative thereof,
c) reducing the compound of Formula IV or its stereoisomers thereof to obtain the compound of Formula I or stereoisomers thereof, and
d) isolating the compound of Formula I or its stereoisomers, and salts thereof, from the reaction mixture thereof.

A fourth aspect of the present invention provides a process for the preparation of a substituted 1,3-oxathiolane of Formula I or its stereoisomers, and salts thereof,

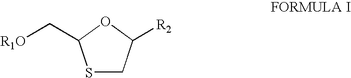

wherein R₁ is hydrogen, alkyl or aryl, and R₂ is an optionally substituted purine or pyrimidine base or an analogue or derivative thereof,
wherein the process comprises,
a) reacting a compound of Formula III or its stereoisomers thereof,

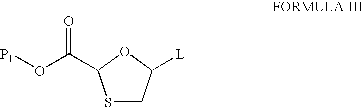

wherein P₁ is hydrogen or a protecting group and L is

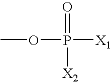

wherein X₁ and X₂ are same or different and are as defined earlier, with an optionally substituted purine or pyrimidine base or an analogue or derivative thereof, to obtain a compound of Formula IV or its stereoisomers thereof,

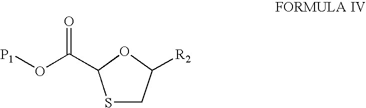

wherein P₁ is hydrogen or a protecting group and R₂ is an optionally substituted purine or pyrimidine base or an analogue or derivative thereof,
b) reducing the compound of Formula IV or its stereoisomers thereof to obtain the compound of Formula I or stereoisomers thereof, and
c) isolating the compound of Formula I or its stereoisomers, and salts thereof, from the reaction mixture thereof.

The compound of Formula V or its stereoisomers thereof, which are used as the starting materials, can be prepared according to the methods provided in U.S. Pat. No. 5,663,320 or Tetrahedron Letters, (2005), 46:8535-8538. The compound of Formula V may be used as a single isomer or as a mixture of two or more isomers. The compound of Formula V is reacted with a compound of Formula VI. The compound of Formula VI is preferably diphenylchlorophosphate or diphenylphosphinic chloride. The reaction is carried out in the presence of an organic solvent and a base. The organic solvent is selected from a group consisting of aliphatic hydrocarbons, aromatic hydrocarbons, halogenated hydrocarbon, nitriles, amides, esters, and ketones. The organic solvent is preferably a halogenated hydrocarbon. The base is preferably a secondary amine or a tertiary amine. The secondary amine is preferably diisopropylamine, dicyclohexylamine, 2,2,6,6-tetramethylethylpiperidine or 1,1,3,3-tetramethylguanidine. The tertiary amine is preferably diisopropylethylamine, triethylamine or tributylamine. A catalytic quantity of a dialkylaminopyridine can also be added to the reaction mixture. The reaction can be carried out at a temperature of about −50° to about 10° C. The formation of the compound of Formula III or its stereoisomers thereof can be facilitated by stirring.

The compound of Formula III or its stereoisomers thereof, can be isolated from the reaction mixture or directly used in the subsequent step without isolation. Preferably the compound of Formula III or its stereoisomers thereof are not isolated from the reaction mixture. The compound of Formula III or its stereoisomers thereof, are reacted with an optionally substituted purine or pyrimidine base or an analogue or derivative thereof, in the absence of a Lewis acid catalyst. The purine or pyrimidine base or an analogue or derivative thereof is preferably selected from the group consisting of:

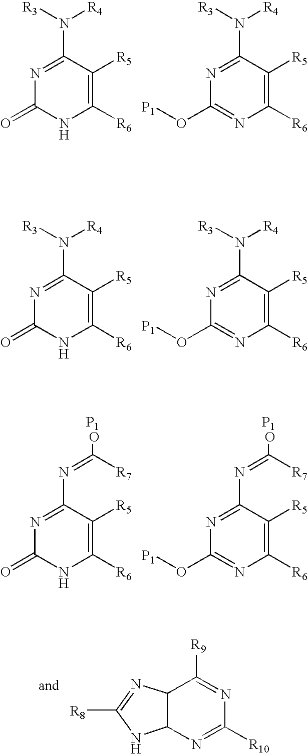

wherein P₁ is a protecting group, R₃ and R₄ are independently selected from the group consisting of hydrogen, hydroxyl, amino, and optionally substituted C₁₋₆ alkyl, C₁₋₆ alkenyl, C₁₋₆ alkynyl, C₁₋₆ acyl or aracyl; R₅ and R₆ are independently selected from the group consisting of hydrogen, halogen, hydroxyl, amino, cyano, carboxy, carbamoyl, alkoxycarbonyl, hydroxymethyl, trifluoromethyl, thioaryl, and optionally substituted C₁₋₆ alkyl, C₁₋₆ alkenyl, C₁₋₆ alkynyl, or C₁₋₁₀ acyloxy; R₇ is C₁₋₆ alkyl, C₁₋₆ alkenyl, or C₁₋₆ alkynyl; R₈ is selected from the group consisting of hydrogen, hydroxy, alkoxy, thiol, thioalkyl, optionally substituted amino, halogen, cyano, carboxy, alkoxycarbonyl, carbamoyl, and optionally substituted C₁₋₆ alkyl, C₁₋₆ alkenyl, C₁₋₆ alkynyl, or C₁-C₁₀ acyloxy; and R₉ and R₁₀ is selected from the group consisting of hydrogen, hydroxy, alkoxy, optionally substituted amino, halogen, azido, and optionally substituted C₁₋₆ alkyl, C₁₋₆ alkenyl, C₁₋₆ alkynyl, or C₁-C₁₀ acyloxy.

The reaction is carried out in the presence of an organic solvent selected from the group consisting of aliphatic hydrocarbons, aromatic hydrocarbons, halogenated hydrocarbon, nitriles, amides, esters, and ketones, preferably at reflux temperature conditions. The reaction is carried out for about 10 minutes to about 100 hours. The reaction may be carried out in the presence of a base. The base is preferably a secondary amine or a tertiary amine. The secondary amine is preferably diisopropylamine, dicyclohexylamine, 2,2,6,6-tetramethylethylpiperidine or 1,1,3,3-tetramethylguanidine. The tertiary amine is preferably diisopropylethylamine, triethylamine or tributylamine.

The compound of Formula IV or its stereoisomers so obtained can be isolated from the reaction mixture or directly used in the subsequent step without isolation. The compound of Formula IV or its stereoisomers are preferably isolated from the reaction mixture. The compound of Formula IV or its stereoisomers are optionally subjected to purification to remove chemical impurities and/or undesired isomers. The protecting groups, if any, present in the compound of Formula IV are removed and the deprotected compound is reduced to obtain the compound of Formula I or its stereoisomers. The reduction is carried out by using a reducing agent. The reducing agent can be, for example, sodium borohydride, lithium aluminium hydride or lithium borohydride. The compound of Formula I or its stereoisomers can be further purified by salt formation, crystallization, isomer separation or chromatographic methods or a combination thereof.

A fifth aspect of the present invention provides a process for the preparation of lamivudine of Formula I (A) or a compound of Formula I (C), or mixtures thereof

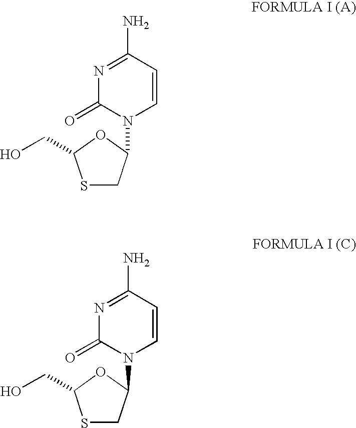

wherein the process comprises,
a) reacting a compound of Formula III (A) or Formula III (B), or mixtures thereof,

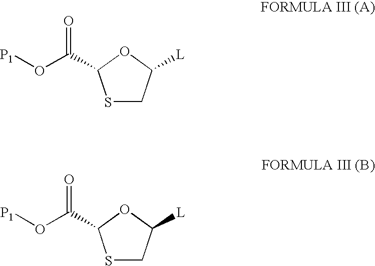

wherein P₁ is a chiral auxiliary and L is

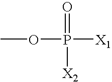

wherein X₁ and X₂ are same or different and are as defined earlier, with cytosine, wherein the amino or hydroxy, or both the groups of said cytosine are optionally protected with protecting groups, to obtain a compound of Formula IV (A) or Formula IV (B), or mixtures thereof

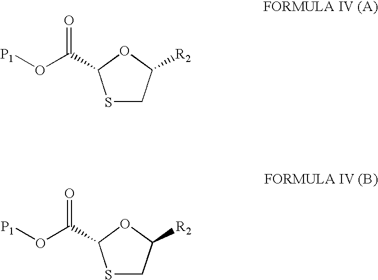

wherein P₁ is a chiral auxiliary and R₂ is cytosine, wherein the amino or hydroxy, or both the groups of said cytosine are optionally protected with protecting groups,
b) reducing the compound of Formula IV (A) or Formula IV (B), or mixtures thereof, to obtain lamivudine of Formula I (A) or the compound of Formula I (C), or mixtures thereof, and
c) isolating lamivudine of Formula I (A) or the compound of Formula I (C), or mixtures thereof, from the reaction mixture thereof.

The compound of Formula III (A) or Formula III (B), or mixtures thereof, which are used as the starting materials, can be prepared by reacting a compound of Formula V (A) or Formula V (B), or mixtures thereof,

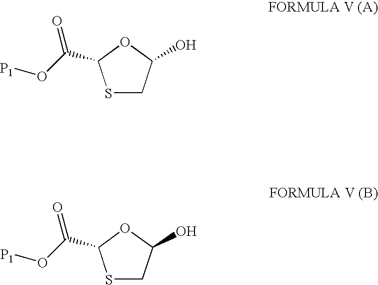

wherein P₁ is a chiral auxiliary, with a compound of Formula VI,

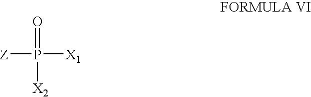

wherein X₁ and X₂ are same or different and are as defined earlier, and Z is halogen. The compound of Formula VI is preferably diphenylchlorophosphate or diphenylphosphinic chloride. The compound of Formula V (A) or Formula V (B), or mixtures thereof can be prepared according to the methods provided in U.S. Pat. No. 5,663,320, or Tetrahedron Letters, (2005), 46:8535-8538. The chiral auxiliary P₁ of the compound of Formula V (A) or Formula V (B), or mixtures thereof, is preferably an L-menthyl group. The reaction of the compound of Formula V (A) or Formula V (B), or mixtures thereof with the compound of Formula VI can be carried out in the presence of an organic solvent and a base. The organic solvent is selected from a group consisting of aliphatic hydrocarbons, aromatic hydrocarbons, halogenated hydrocarbon, nitriles, amides, esters, and ketones. The organic solvent is preferably a halogenated hydrocarbon. The base is preferably a secondary amine or a tertiary amine. The secondary amine is preferably diisopropylamine, dicyclohexylamine, 2,2,6,6-tetramethylethylpiperidine or 1,1,3,3-tetramethylguanidine. The tertiary amine is preferably diisopropylethylamine, triethylamine or tributylamine. A catalytic quantity of a dialkylaminopyridine can also be added to the reaction mixture. The reaction is preferably carried out at a temperature of about −50° to about 10° C. The formation of the compound of Formula III (A) or Formula III (B), or mixtures thereof, can be accompanied by stirring.

The compound of Formula III (A) or Formula III (B), or mixtures thereof, is reacted with cytosine, wherein the amino or hydroxy, or both the groups of said cytosine are optionally protected with protecting groups. The cytosine is preferably protected with acetyl and/or silyl protecting groups. The reaction is carried out in the presence or absence of a Lewis acid catalyst, preferably in the absence of any Lewis acid catalyst. The reaction is carried out in the presence of an organic solvent selected from the group consisting of aliphatic hydrocarbons, aromatic hydrocarbons, halogenated hydrocarbon, nitriles, amides, esters, and ketones, preferably at reflux temperature conditions. The reaction is carried out for about 10 minutes to about 100 hours. The reaction may be carried out in the presence of a base. The base is preferably a secondary amine or a tertiary amine. The secondary amine is preferably diisopropylamine, dicyclohexylamine, 2,2,6,6-tetramethylethylpiperidine or 1,1,3,3-tetramethylguanidine. The tertiary amine is preferably diisopropylethylamine, triethylamine or tributylamine.

The compound of Formula IV (A) or Formula IV (B), or mixtures thereof can be isolated from the reaction mixture or directly used in the subsequent step without isolation.

The compound of Formula IV (A) or Formula IV (B), or mixtures thereof are preferably isolated from the reaction mixture. A deprotection step may be carried out to remove the protecting groups, if any, present in R₂ of the compound of Formula IV (A) or Formula IV (B), or mixtures thereof. The compound of Formula IV (A) or Formula IV (B), or mixtures thereof are optionally subjected to purification to remove chemical impurities and/or undesired isomers. The compound of Formula IV (A) or Formula IV (B), or mixtures thereof are reduced to obtain lamivudine of Formula I (A) or the compound of Formula I (C), or mixtures thereof. The reduction is carried out by using a reducing agent. The reducing agent can be, for example, sodium borohydride, lithium aluminium hydride, lithium borohydride, lithium-tri-ethyl borohydride or lithium-tri-sec-butyl borohydride. Lamivudine of Formula I (A) or the compound of Formula I (C), or mixtures thereof can be further purified by salt formation, crystallization, isomer separation or chromatographic methods or a combination thereof.

A sixth aspect of the present invention provides a process for the preparation of lamivudine of Formula I (A),

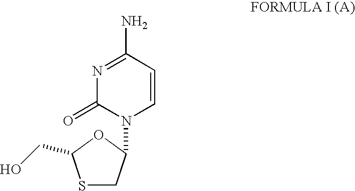

wherein the process comprises,
a) reacting a compound of Formula III (C),

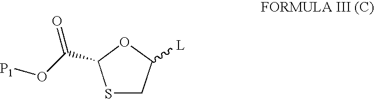

wherein P₁ is a chiral auxiliary and L is

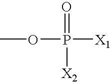

wherein X₁ and X₂ are same or different and are as defined earlier,
with cytosine, wherein the amino or hydroxy, or both the groups of said cytosine are optionally protected with protecting groups, to obtain a compound of Formula IV (C),

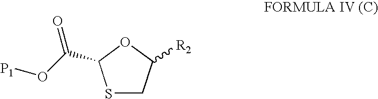

wherein P₁ is a chiral auxiliary and R₂ is cytosine, wherein the amino or hydroxy, or both the groups of said cytosine are optionally protected with protecting groups,
b) separating a compound of Formula IV (A) from the reaction mixture thereof,

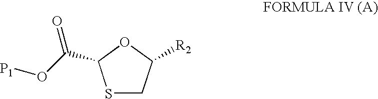

wherein P₁ is a chiral auxiliary and R₂ is cytosine, wherein the amino or hydroxy, or both the groups of said cytosine are optionally protected with protecting groups,
c) reducing the compound of Formula IV (A) to obtain lamivudine of Formula I (A), and
d) isolating lamivudine of Formula I (A) from the reaction mixture thereof.

The compound of Formula III (C), which is used as the starting material, can be prepared according to the method disclosed in the previous aspect of the present invention. The chiral auxiliary P₁ of the compound of Formula III (C) is preferably an L-menthyl group. The compound of Formula III (C) is reacted with cytosine, wherein the amino or hydroxy, or both the groups of said cytosine are optionally protected with protecting groups. The cytosine is preferably protected with acetyl and/or silyl protecting groups. The reaction is carried out in the presence or absence of a Lewis acid, preferably in the absence of any Lewis acid catalyst. The reaction is carried out in the presence of an organic solvent selected from the group consisting of aliphatic hydrocarbons, aromatic hydrocarbons, halogenated hydrocarbon, nitriles, amides, esters, and ketones, preferably at reflux temperature conditions. The reaction is carried out for about 10 minutes to about 100 hours. The reaction may be carried out in the presence of a base. The base is preferably a secondary amine or a tertiary amine. The secondary amine is preferably diisopropylamine, dicyclohexylamine, 2,2,6,6-tetramethylethylpiperidine or 1,1,3,3-tetramethylguanidine. The tertiary amine is preferably diisopropylethylamine, triethylamine or tributylamine.

The compound of Formula IV (C) so obtained may be subjected to deprotection to remove the silyl protecting groups, if any, present in the cytosine group. The compound of Formula IV (C) is isolated from the reaction mixture by concentrating the reaction mixture. The compound of Formula IV (A) is separated from the compound of Formula IV (C) by selective crystallization methods, chiral chromatographic methods or by chiral salt formation, or a combination thereof. The compound of Formula IV (A) is preferably separated by treating the compound of Formula IV (C) with a solvent, which selectively dissolves the undesired isomers while the compound of Formula IV (A) is partially or completely insoluble in said solvent. The treatment with the solvent may be carried out once or more than once to achieve desired optical purity. The solvent is preferably a C₁₋₃ alkanol or an aliphatic ester, or a mixture thereof, more preferably methanol or isopropyl acetate, or a mixture thereof. The compound of Formula IV (A) is isolated from the reaction mixture by filtration after treating with the solvent.

The compound of Formula IV (A) is deprotected to remove the acetyl protecting groups, if any, present in R₂ of the compound of Formula IV (A). The deprotected compound is reduced to obtain lamivudine of Formula I (A). The reduction is carried out by using a reducing agent. The reducing agent can be, for example, sodium borohydride, lithium aluminium hydride, lithium borohydride, lithium-tri-ethyl borohydride or lithium-tri-sec-butyl borohydride. The reducing agent is preferably sodium borohydride. The reduction is carried out in the presence of a phosphate or borate buffer. The buffer is preferably dipotassium hydrogen phosphate. Lamivudine of Formula I (A) can be further purified by salt formation, crystallization, or chromatographic methods, or a combination thereof.

The lamivudine so obtained is preferably further purified by salt formation by treating with salicylic acid in the presence of an organic solvent, or a mixture of water and an organic solvent. The lamivudine salicylate so obtained is treated with a base in the presence of an organic solvent, or a mixture of water and an organic solvent. The base is preferably an amine, more preferably a tertiary amine. The lamivudine so obtained can be purified further by charcoal treatment in the presence of a C₁₋₃ alkanol. The lamivudine so obtained has a chemical purity of about 99% or above and a chiral purity of about 99.5% or above, preferably of about 99.8% or above.

A seventh aspect of the present invention provides a process for the preparation of a substituted 1,3-oxathiolane of Formula I or its stereoisomers, and salts thereof,

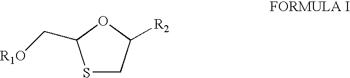

wherein R₁ is hydrogen, alkyl or aryl, and R₂ is an optionally substituted purine or pyrimidine base or an analogue or derivative thereof,
wherein the process comprises,
a) reacting a compound of Formula III or its stereoisomers thereof,

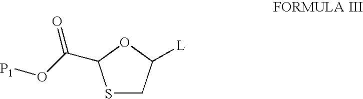

wherein P₁ is hydrogen or a protecting group and L is a leaving group, with proviso that the leaving group is not halo, cyano or substituted sulfonyl,
with a substituted or unsubstituted purine or pyrimidine base or an analogue or derivative thereof,
in the absence of a Lewis acid catalyst, to obtain a compound of Formula IV or its stereoisomers thereof,

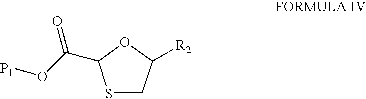

wherein P₁ is hydrogen or a protecting group and R₂ is an optionally substituted purine or pyrimidine base or an analogue or derivative thereof,
b) reducing the compound of Formula IV or its stereoisomers thereof to obtain the compound of Formula I or stereoisomers thereof, and
c) isolating the compound of Formula I or its stereoisomers, and salts thereof, from the reaction mixture thereof.

The compound of Formula III or its stereoisomers thereof, which are used as the starting materials, can be prepared according to the methods provided in U.S. Pat. No. 5,663,320 or U.S. Pat. No. 6,175,008, or according to the methods disclosed in the previous aspects of the present invention. The compound of Formula III may be used as a single isomer or as a mixture of two or more isomers. The compound of Formula III is reacted with a substituted or unsubstituted purine or pyrimidine base or an analogue or derivative thereof. The purine or pyrimidine base or an analogue or derivative thereof is preferably selected from the group described hereinbefore.

The reaction is effected without the addition of any Lewis acid catalyst and the is carried out in the presence of an organic solvent selected from the group consisting of aliphatic hydrocarbons, aromatic hydrocarbons, halogenated hydrocarbon, nitriles, amides, esters, and ketones. The reaction is carried out for about 10 minutes to about 100 hours preferably at reflux temperature conditions. The compound of Formula IV or its stereoisomers can be isolated from the reaction mixture or directly used in the subsequent step without isolation. The compound of Formula IV or its stereoisomers are optionally subjected to purification to remove chemical impurities and/or undesired isomers. The protecting groups, if any, present in the compound of Formula IV are removed and the deprotected compound is reduced to obtain the compound of Formula I or its stereoisomers. The reduction is carried out by using a reducing agent. The reducing agent can be, for example, sodium borohydride, lithium aluminium hydride or lithium borohydride. The compound of Formula I or its stereoisomers can be further purified by salt formation, crystallization, isomer separation or chromatographic methods or a combination thereof.

While the present invention has been described in terms of its specific embodiments, certain modifications and equivalents will be apparent to those skilled in the art and are intended to be included within the scope of the present invention.

### EXAMPLES

### Example 1

### Preparation of (1R,2S,5R)-2-isopropyl-5-methylcyclohexyl (2R,5S)-5-[4-(acetylamino)-2-oxopyrimidin-1(2H)-yl]-1,3-oxathiolane-2-carboxylate

Step A: Methane sulfonic acid (0.5 mL) was added to a mixture of N-acetyl cytosine (100 g), hexamethyldisilazane (150 mL) and toluene (250 mL). The reaction mixture was refluxed till a clear solution was obtained.
Step B: Dimethylaminopyridine (9.5 g) was added to a solution of (1R,2S,5R)-2-isopropyl-5-methylcyclohexyl (2R,5R)-5-hydroxy-1,3-oxathiolane-2-carboxylate (190 g) and diphenylphosphinic chloride (190 g) in dichloromethane (600 mL) at 0° C. Diisopropylethylamine (119 g) was subsequently added slowly to the reaction mixture at −20° to −10° C. and stirred for 1 h at −20° to −10° C.
Step C: Triethylamine (86 g) was added to the solution obtained in Step A, followed by the addition of the reaction mixture obtained in Step B at reflux temperature. The reaction mixture was refluxed for 6 to 7 h, and cooled to about 25° C. The reaction mixture was poured into a mixture of methanol (500 mL), concentrated hydrochloric acid (200 mL) and water (1 L) at 15° to 20° C. The reaction mixture was stirred for 5 to 10 minutes, allowed to settle and the organic layer was washed with water (500 mL). The organic layer was concentrated and isopropyl acetate (1 L) was added to the residue. The mixture was stirred for 5 to 6 h, filtered and washed with isopropyl acetate (200 mL). The washed solid was dried under vacuum for 5 h at 45° to 50° C. to obtain the title compound.

Yield: 68 g

### Example 2

### Preparation of (1R,2S,5R)-2-isopropyl-5-methylcyclohexyl (2R,5S)-5-[4-(acetylamino)-2-oxopyrimidin-1(2H)-yl]-1,3-oxathiolane-2-carboxylate

Step A: Methane sulfonic acid (0.5 mL) was added to a mixture of N-acetyl cytosine (100 g), hexamethyldisilazane (150 mL) and toluene (250 mL). The reaction mixture was refluxed till a clear solution was obtained.
Step B: Dimethylaminopyridine (9.5 g) was added to a solution of (1R,2S,5R)-2-isopropyl-5-methylcyclohexyl (2R,5R)-5-hydroxy-1,3-oxathiolane-2-carboxylate (190 g) and diphenylchloro phosphate (215 g) in dichloromethane (600 mL). Diisopropylethylamine (145 g) was subsequently added slowly to the reaction mixture at 0° to 5° C. and stirred for 1 h at 0° to 5° C.
Step C: Triethylamine (86 g) was added to the solution obtained in Step A, followed by the addition of the reaction mixture obtained in Step B at reflux temperature. The reaction mixture was refluxed for 4 to 5 h, and cooled to 30° to 35° C. Methanol (100 mL) was added to the reaction mixture, filtered and the organic layer was washed with water (2×1 L). The organic layer was concentrated and isopropyl acetate (1 L) was added to the residue. The mixture was stirred for 5 to 6 h, filtered and washed with isopropyl acetate (200 mL). The solid obtained was dried under vacuum at 45° to 50° C. to obtain the title compound.

Yield: 80 g

### Example 3

### Preparation of (1R,2S,5R)-2-isopropyl-5-methylcyclohexyl(2R,5S)-5-(4-amino-2-oxopyrimidin-1(2H)-yl)-1,3-oxathiolane-2-carboxylate

(1R,2S,5R)-2-isopropyl-5-methylcyclohexyl(2R,5S)-5-[4-(acetylamino)-2-oxopyrimidin-1(2H)-yl]-1,3-oxathiolane-2-carboxylate (100 g) obtained from Example 2 was suspended in methanol (600 mL) at about 25° C. Methane sulfonic acid (29.4 g) was added drop-wise to the suspension in 15 to 20 minutes at 25° to 30° C. and stirred for 4 h at about 25° C. The reaction mixture was added slowly to a mixture of dichloromethane (1 L) and aqueous sodium bicarbonate solution (28 g of sodium bicarbonate dissolved in 1.2 L of water). The reaction mixture was stirred for 5 to 10 minutes and allowed to settle. The layers were separated and the organic layer was concentrated. Hexane (500 mL) was added to the residue and stirred for 2 h. The solid obtained was filtered and washed with hexane (100 mL), followed by isopropyl acetate (200 mL). The washed solid was dried at 45° to 50° C. to obtain the title compound.

Yield: 80 g

PLC Purity: 98%

### Example 4

### Preparation of Lamivudine Salicylate

Dipotassium hydrogen orthophosphate (205.5 g) was added to deionised water (423 mL) and stirred at 25° to 30° C. to obtain a solution. The solution was cooled to 17° to 22° C., followed by the addition of denaturated spirit (900 mL) at the same temperature and stirred for 5 minutes. (1R,2S,5R)-2-isopropyl-5-methylcyclohexyl (2R,5S)-5-(4-amino-2-oxopyrimidin-1(2H)-yl)-1,3-oxathiolane-2-carboxylate (150 g) was added to the mixture at 17° to 22° C. and stirred for 30 minutes at 18° to 20° C. Sodium borohydride solution was added slowly to the reaction mixture over a period of 2 to 3 h at 18° to 20° C. (Preparation of sodium borohydride solution: Sodium hydroxide (0.75 g) was dissolved in deionised water (143 mL). Sodium borohydride (30 g) was added to the solution at 20° to 35° C., stirred at 20° to 35° C. to obtain a solution and cooled to 17° to 22° C.). The reaction mixture was stirred for 6 h at 18° to 22° C. and the reaction mixture was allowed to settle at 18° to 25° C. The organic layer was separated and denaturated spirit (150 mL) was added to the aqueous layer at 18° to 25° C. The reaction mixture was stirred for 15 minutes at the same temperature and allowed to settle. The organic layer was separated and combined with the previously obtained organic layer. The pH of the combined organic layer was adjusted to 6.0 to 6.5 with dilute hydrochloric acid (20 mL; prepared by mixing 10 mL of concentrated hydrochloric acid with 10 mL of deionised water) at 18° to 25° C., followed by stirring for 10 minutes at the same temperature. The pH of the reaction mixture was adjusted to 8.0 to 8.5 with aqueous sodium hydroxide solution (28 mL; prepared by dissolving 2.1 g of sodium hydroxide in 27 mL of deionised water) at 18° to 25° C. The reaction mixture was concentrated under vacuum at about 55° C. till the residual volume was about 375 mL. Deionised water (300 mL) was added to the concentrated reaction mixture at 25° to 30° C. and stirred for 10 minutes. The reaction mixture was washed with toluene (2×150 mL) at 25° to 30° C. and the toluene layer was extracted with deionised water (150 mL) at 25° to 30° C. The aqueous layers were combined and salicylic acid (57 g) was added at 25° to 30° C. Deionised water (150 mL) was added to the reaction mixture and heated to 78° to 82° C. to get a clear solution. The reaction mixture was cooled to 25° to 30° C. over a period of 2 h and stirred at the same temperature for 4 h. The reaction mixture was further cooled to 10° to 15° C. and stirred for 2 h at 10° to 15° C. The solid was filtered, washed with deionised water (150 mL) and dried by suction. The solid so obtained was washed with methanol (90 mL, pre-cooled to 5° to 10° C.) and dried at 45° to 50° C. in hot air oven to obtain the title compound.

Yield: 132 g

### Example 5

### Preparation of Lamivudine

Lamivudine salicylate (120 g) was added to a mixture of ethyl acetate (720 mL) and water (6 mL) at 25° to 35° C. The reaction mixture was heated to 45° to 50° C., followed by the addition of triethylamine (104.76 g) over 30 minutes at 45° to 50° C. The reaction mixture was stirred for 4 h at the same temperature and cooled to 25° to 30° C. The reaction mixture was stirred for further 30 minutes at 25° to 30° C., filtered and dried by suction. The solid obtained was washed with ethyl acetate. Ethyl acetate (600 mL) was added to the washed solid and heated to 50° to 55° C. The mixture was stirred at 50° to 55° C. for 15 minutes, cooled to 25° to 30° C. and stirred for further 30 minutes. The solid was filtered at 25° to 30° C., washed with ethyl acetate (60 mL) and dried under vacuum at 45° to 50° C. to obtain the title compound.

Yield: 68.5 g

### Example 6

### Purification of Lamivudine

Lamivudine (60 g) obtained from Example 4 was added to absolute alcohol (1.2 L) at 25° to 35° C. The reaction mixture was heated to 75° to 78° C. and stirred to obtain a solution. Activated carbon (6 g) was added to the solution so obtained at 75° to 78° C., stirred for 30 minutes at the same temperature and filtered through Celite bed at the same temperature. The carbon bed was washed with absolute alcohol (60 mL; preheated to 75° to 76° C.) and the reaction mixture was concentrated under vacuum to obtain a volume of about 300 mL. The concentrated reaction mixture was heated to 74° to 76° C., stirred for 15 minutes and cooled to 20° to 25° C. in 1 h time. The reaction mixture was further cooled to 5° to 10° C. in 1 h time and stirred for 30 minutes. The solid was filtered, washed with absolute alcohol (30 mL, pre-cooled to 5° to 10° C.) and dried under vacuum at 50° to 55° C. to obtain the title compound.

Yield: 53 g

HPLC Purity: 99.0%

Chiral Purity: 99.8%

We claim:

1. A compound of Formula III or its stereoisomers thereof, 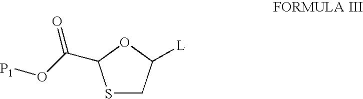
wherein P₁ is hydrogen or a protecting group and L is 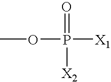
wherein X₁ and X₂ are same or different and selected from the group consisting of hydrogen, optionally substituted straight chain or cyclic alkyl, optionally substituted or unsubstituted aryl, optionally substituted alkyloxy, optionally substituted aryloxy and optionally substituted aralkyl.

2. A compound according to claim 1, wherein X₁ and X₂ are optionally substituted aryl or aryloxy groups.

3. A compound according to claim 1, wherein P₁ is a chiral auxiliary.

4. A process for the preparation of compound of Formula III or its stereoisomers thereof, 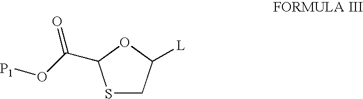
wherein P₁ is hydrogen or a protecting group and L is 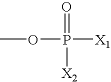
wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl,
wherein the process comprises a step of reacting a compound of Formula V, 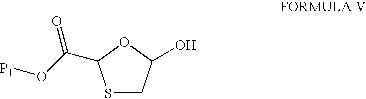
wherein P₁ is hydrogen or a protecting group, with a compound of Formula VI, 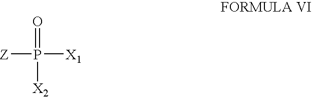
wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl, and Z is halogen, to obtain the compound of Formula III or its stereoisomers thereof.

5. The use of a compound of Formula III in a process for the preparation of a substituted 1,3-oxathiolane of Formula I or its stereoisomers, and salts thereof, 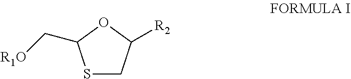
wherein R₁ is hydrogen, alkyl or aryl, and R₂ is an optionally substituted purine or pyrimidine base or an analogue or derivative thereof,
wherein the process comprises,
reacting a compound of Formula V, 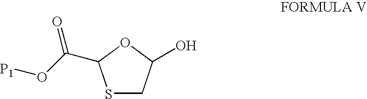
wherein P₁ is hydrogen or a protecting group, with a compound of Formula VI, 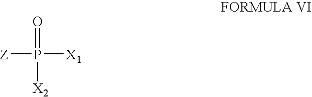
wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl, and Z is halogen, to obtain a compound of Formula III or its stereoisomers thereof, 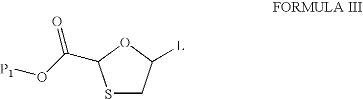
wherein P₁ is hydrogen or a protecting group and L is 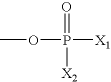
wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl,
b) reacting a compound of Formula III or its stereoisomers thereof, with an optionally substituted purine or pyrimidine base or an analogue or derivative thereof, to obtain a compound of Formula IV or its stereoisomers thereof, 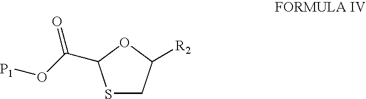
wherein P₁ is hydrogen or a protecting group and R₂ is an optionally substituted purine or pyrimidine base or an analogue or derivative thereof,
c) reducing the compound of Formula IV or its stereoisomers thereof to obtain the compound of Formula I or stereoisomers thereof, and
d) isolating the compound of Formula I or its stereoisomers, and salts thereof, from the reaction mixture thereof.

6. The use of a compound of Formula III(A) or III (B) in a process for the preparation of lamivudine of Formula I (A) or a compound of Formula I (C), or mixtures thereof 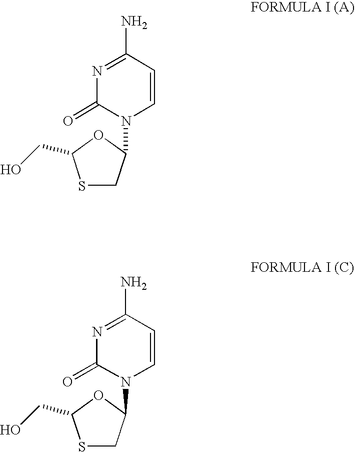
wherein the process comprises,
reacting a compound of Formula III (A) or Formula III (B), or mixtures thereof, 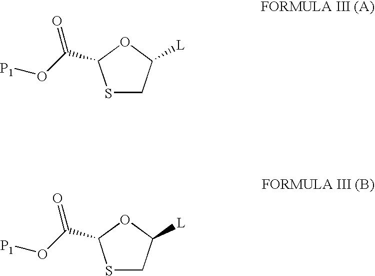
wherein P₁ is a chiral auxiliary and L is 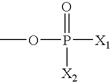
wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl,
with cytosine, wherein the amino or hydroxy, or both the groups of said cytosine are optionally protected with protecting groups, to obtain a compound of Formula IV (A) or Formula IV (B), or mixtures thereof 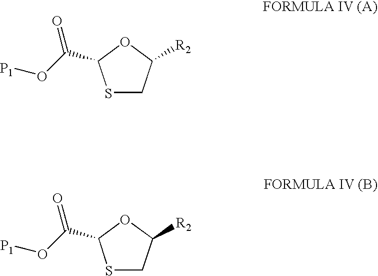
wherein P₁ is a chiral auxiliary and R₂ is cytosine, wherein the amino or hydroxy, or both the groups of said cytosine are optionally protected with protecting groups,
b) reducing the compound of Formula IV (A) or Formula IV (B), or mixtures thereof, to obtain lamivudine of Formula I (A) or the compound of Formula I (C), or mixtures thereof, and
c) isolating lamivudine of Formula I (A) or the compound of Formula I (C), or mixtures thereof, from the reaction mixture thereof.

7. A process for the preparation of lamivudine of Formula I (A), 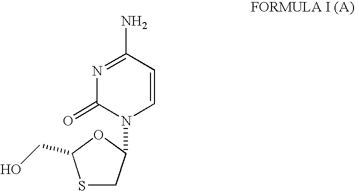
wherein the process comprises,
a) reacting a compound of Formula III (C), 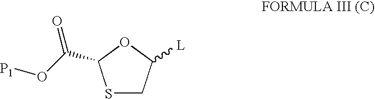
wherein P₁ is a chiral auxiliary and L is 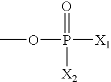
wherein X₁ and X₂ are same or different and selected from the group consisting of hydrogen, optionally substituted straight chain or cyclic alkyl, optionally substituted aryl, optionally substituted alkyloxy, optionally substituted aryloxy and optionally substituted aralkyl,
with cytosine, wherein the amino or hydroxy, or both the groups of said cytosine are optionally protected with protecting groups, to obtain a compound of Formula IV (C), 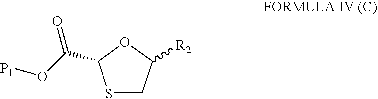
wherein P₁ is a chiral auxiliary and R₂ is cytosine, wherein the amino or hydroxy, or both the groups of said cytosine are optionally protected with protecting groups,
b) separating a compound of Formula IV (A) from the reaction mixture thereof, 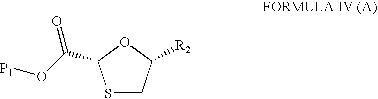
wherein P₁ is a chiral auxiliary and R₂ is cytosine, wherein the amino or hydroxy, or both the groups of said cytosine are optionally protected with protecting groups,
c) reducing the compound of Formula IV (A), and
d) isolating lamivudine of Formula I (A) from the reaction mixture thereof.

8. Use of the compound of claim 1 for preparing lamivudine.
